(19) United States
(12) Patent Application Publication
(10) Pub. No.: US 2025/0089981 A1
(43) Pub. Date: Mar. 20, 2025

(54) EXTENDED MULTIPLEXED INTER-INTEGRATED CIRCUIT BUS FOR DIGITAL ENDOSCOPY

(71) Applicant: Magnisity Ltd., Hod-HaSharon (IL)

(72) Inventors: Ron BARAK, Tel-Aviv (IL); Benjamin GREENBURG, Hod-HaSharon (IL)

(73) Assignee: Magnisity Ltd., Hod-HaSharon (IL)

(21) Appl. No.: 18/882,970

(22) Filed: Sep. 12, 2024

Related U.S. Application Data
(60) Provisional application No. 63/538,290, filed on Sep. 14, 2023.

(51) Int. Cl. A61B 1/00

(52) U.S. Cl. CPC A61B 1/00006

(57) ABSTRACT

A system and method for communication in an extended multiplexed inter-integrated circuit bus configuration, in which an inter-integrated circuit bus including a plurality of wires, a plurality of target devices, each includes at least two ports of respective two types; and a controller device, communicatively connected to the plurality of target devices by an extended multiplexed configuration of the inter-integrated circuit bus, wherein a first group of at least two of the target devices are allocated to a first same target address, wherein each of the target devices of the first group is connected to the bus by a different wire configuration of which wire is connected to which port type.

### RELATED APPLICATION(S)

This application claims the benefit of priority under 35 USC § 119 (e) of U.S. Provisional Patent Application No. 63/538,290 filed on Sep. 14, 2023, the contents of which are incorporated by reference as if fully set forth herein in their entirety.

### FIELD AND BACKGROUND OF THE INVENTION

The present disclosure relates to communication of a controller with sensors, and more specifically communication with sensors for digital endoscopy.

An Inter-Integrated Circuit (I2C) digital bus uses two digital signals: SCL (clock), SDA (data) and it enables communication between a master device (which is responsible for generating the clock signal) and one or more slave devices. The clock is generated by the I2C master, and the data is sometimes generated by the master (when the master writes) and sometimes generated by the slave (when the master reads). The I2C bus is open-drain or open-collector, and pull-up resistors (for example, 10 kOhm) are attached to each line. A logic LOW is output by pulling the line to GND, and a logic HIGH is output by releasing the line output (HiZ), so that the pull-up resistor pulls it high.

In order to support multiple slave devices on the same I2C bus, each sensor usually has a unique 7-bit I2C address. When the master initiates communication, it starts by initiating a START condition (indication) on the I2C bus, then writing a READ/WRITE indication bit, followed by the slave address with which it wants to communicate (on the SDA wire). All the slave devices listen on a same I2C bus and respond only when the master addresses their unique 7-bit I2C address. This way a situation where multiple slave devices respond simultaneously is avoided and the master is then able to address each slave device individually. The master terminates communication by writing a STOP condition on the I2C bus.

Known Electromagnetic (EM) localization systems are sometimes used in the medical field for tracking a small catheter inside the body.

In a traditional EM-tracked catheter setting, a catheter would have one or more micro coils placed at its tip. For example, three micro-sized coils need to be placed at the tip of the catheter, traditionally in an orthogonal manner, and wires need to be pulled outside to an external DSP. In some specialized systems a single coil is used rather than three orthogonal coils, but this requires a specialized EM field generator to generate many different unique fields, and in addition it can only provide localization of up to five degrees-of-freedom (with roll angle missing). In general, one-coil systems are inferior in terms of accuracy and overall stability. The EM field generator would generate a high-frequency (>1 kHz, for example) alternating magnetic field which would induce electromotive force (EMF) on the catheter's coils (by Faraday's law of induction). The coils are connected through wires to an external digital signal processor (DSP) unit which is responsible for amplifying the induced voltage, then sampling it, for example by using an analog-to-digital (A2D) converter. Every such coil requires a separate processing channel with a dedicated high-quality amplifier and A2D input. In addition, good signal-noise ratio (SNR) is hard to maintain; the tiniest noise might be amplified and obscure the signal of interest. For example, the wires connecting the coils to the DSP might form a loop through which some magnetic flux flows and which therefore picks up some amount of parasitic, undesired signal from the transmitted fields. This requires the wires to be wound across the catheter in a twisted pair fashion. In addition to the wires, the connector which connects the catheter to the DSP might also form an undesired loop which is able to pick up some amount of parasitic signal. A traditional EM catheter therefore involves a complex design, with a special, complicated, and expensive, complementary DSP unit. The complexity of such a system grows almost linearly with the number of desired sensors (the number of coils, number of twisted-pair wires, number of DSP input channels which include more expensive amplifiers and A2D).

Additional background art includes U.S. Pat. No. 11,712,309, disclosing an EM shape sensor is described which consists of a sensor-array made of multiple discrete digital 3D magnetometers assembled on a Flexible Printed Circuit (FPC). The sensor-array may be embedded in an endoscope (or other tubular device) to enable EM shape sensing of that endoscope.

### SUMMARY OF THE INVENTION

An aspect of some embodiments of the present disclosure provides a system for communication in an extended multiplexed inter-integrated circuit bus configuration, the system includes an inter-integrated circuit bus including a plurality of wires; a plurality of target devices, each includes at least two ports of respective two types; and a controller device, communicatively connected to the plurality of target devices by an extended multiplexed configuration of the inter-integrated circuit bus, wherein a first group of at least two of the target devices are allocated to a first same target address, wherein each of the target devices of the first group is connected to the bus by a different wire configuration of which wire is connected to which port type.

Optionally, the controller device is configured to: initiate a start indication recognizable by a first target device of the first group, and unrecognizable by the other target devices of the first group, based on the wire configuration by which the first device is connected to the bus; and transmit a communication along with an indication of the first same target address of the first group.

Optionally, a second group of at least two of the target devices are allocated to a second same target address, wherein each of the target devices of the second group is connected to the bus by a different wire configuration of which wire is connected to which port type, wherein the controller device is configured to: initiate a start indication recognizable by a first target device of the first group and a first target device of the second group, and unrecognizable by the other target devices of the first and second groups, based on the wire configuration by which the first devices is connected to the bus; and transmit a communication along with an indication of the same target address of the first group.

Optionally, the target devices of the same group share the same target address and are assembled to the same extended bus, with respective different wire configurations, in which a clock signal wire and a data signal wire are flipped in a first configuration with respect to a second configuration.

Optionally, the target devices comprise digital magnetometer sensors in an electromagnetic curve-tracked (shape and/or position tracked) endoscope.

Optionally, the extended bus has at least three wires, wherein the controller selects two of the three possible wires, designates one wire as clock signal and the second as data signal, and communicates individually with each of a plurality of the target devices which are connected to the selected two wires.

Optionally, the extended bus wires are each pulled-up by a pull-up resistor.

Optionally, the controller communicates in parallel with two or more target devices which are connected through different data signals but which may share a same clock signal.

Another aspect of some embodiments of the present disclosure provides a method for communication in an extended multiplexed inter-integrated circuit bus configuration, the method includes: initiating by a controller device a start indication recognizable by a first target device of a first group of devices, the start indication is unrecognizable by other target devices of the first group, based on a wire configuration by which the first device is connected to the bus, wherein the first group includes at least two target devices that are allocated to a first same target address, wherein each of the target devices of the first group is connected to the bus by a different wire configuration of which wire is connected to which port type, wherein each target device includes at least two ports of respective two types; and transmitting a communication along with an indication of the first same target address of the first group.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an extended multiplexed inter-integrated circuit bus configuration for digital endoscopy, according to some embodiments of the present disclosure;

FIG. 2 is a schematic flowchart illustrating a method for communication in an extended multiplexed inter-integrated circuit bus configuration for digital endoscopy, according to some embodiments of the present disclosure;

FIG. 3 is a schematic illustration of an extended multiplexed inter-integrated circuit bus configuration for digital endoscopy, according to some embodiments of the present disclosure; and

FIG. 4 is a schematic illustration of an extended multiplexed inter-integrated circuit bus configuration for digital endoscopy, according to some embodiments of the present disclosure.

### DESCRIPTION OF SPECIFIC EMBODIMENTS OF THE INVENTION

The present disclosure relates to communication of a controller with sensors, and more specifically communication with sensors for digital endoscopy.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present disclosure describes a system for communication in an extended multiplexed inter-integrated circuit bus configuration for digital endoscopy, the system comprising: an inter-integrated circuit bus including a plurality of wires; a plurality of target devices, each includes at least two ports of respective two types; and a controller device, communicatively connected to the plurality of target devices by an extended multiplexed configuration of the inter-integrated circuit bus, wherein a first group of at least two of the target devices are allocated to a first same target address, wherein each of the target devices of the first group is connected to the bus by a different wire configuration of which wire is connected to which port type, wherein the controller device is configured to: initiate a start indication recognizable by a first target device of the first group, and unrecognizable by the other target devices of the first group, based on the wire configuration by which the first device is connected to the bus; and transmit a communication along with an indication of the first same target address of the first group.

Optionally, a second group of at least two of the target devices are allocated to a second same target address, wherein each of the target devices of the second group is connected to the bus by a different wire configuration of which wire is connected to which port type, wherein the controller device is configured to: initiate a start indication recognizable by a first target device of the first group and a first target device of the second group, and unrecognizable by the other target devices of the first and second groups, based on the wire configuration by which the first devices is connected to the bus; and transmit a communication along with an indication of the same target address of the first group.

In U.S. Pat. No. 11,712,309, an elongated device containing multiple embedded digital magnetometers is disclosed. The digital magnetometers are used to provide shape and/or position and/or curve tracking of the elongated device. The digital magnetometers may communicate over an Inter-Integrated Circuit (I2C) bus, and the bus may be manufactured on an FPC (Flexible Printed Circuit) which may be embedded inside the wall of an endoscope, for example, by wrapping it around the endoscope's working channel.

Some embodiments of the present disclosure provide systems and methods, including an extended multiplexed inter-integrated circuit bus for digital endoscopy. The provided circuit may include an extended multiplexed I2C bus, optionally for use in digital endoscopy, specifically, for electromagnetic (EM) shape and/or curve tracking. In some embodiments, multiple digital components are used, for example, digital magnetometer sensors, which communicate digitally through an I2C bus. In some embodiments, some of the digital components may share a same I2C address. Some aspects of the present disclosure provide a multiplexed I2C bus design, which enables communication with multiple digital components sharing the same I2C address. In some embodiments, these components are embedded in an elongated device of small footprint (for example, a thin endoscope of outer diameter of, for example, 3.5 mm). The multiplexed I2C bus may contain as few as two I2C wires, or three I2C wires, or four I2C wires, which results in a small bus footprint. The bus can be designed on a Flexible Printed Circuit (FPC). In some embodiments, the FPC can be embedded in a thin endoscope, with only a small potential increase in the outer diameter (OD).

It will be appreciated that throughout the present disclosure, the terms “I2C” or “Inter Integrated Circuit” may also mean and/or include similar and/or equivalent and/or improved technologies, such as I3C.

In a standard I2C bus, the I2C controller device may not be able to communicate separately with multiple target devices sharing the same I2C address, because they all may respond to the READ/WRITE requests transmitted from the controller device and received on their shared I2C address. For example, if two digital magnetometer sensors share the same I2C address and respond to the controller's sample READ request they may both simultaneously write their magnetic samples on the I2C bus at the same time, such that the output result will be a bitwise-AND operation (due to the bus's open-drain configuration) between the two sensor outputs, as will be read by the I2C controller, and the controller will not be able to extract each sensor's individual reading.

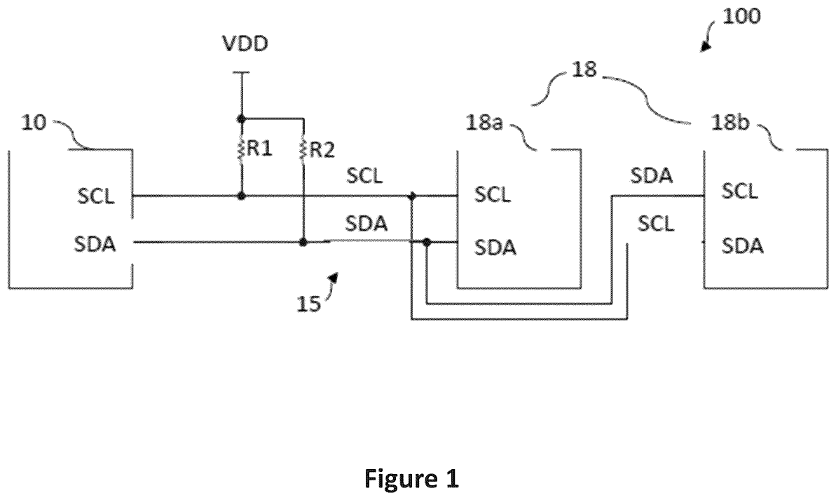

Reference is now made to FIG. 1, which is a schematic illustration of an extended multiplexed inter-integrated circuit bus configuration 100 for digital endoscopy, according to some embodiments of the present disclosure. Bus configuration 100 may include a controller device 10 and I2C target devices 18. According to some embodiments, wires are multiplexed such that each of target devices 18 is connected to a unique selection of two I2C wires, on an extended multiplexed bus 15, for example in order to enable for controller device 10 individual sampling of the sensors in target devices 18.

Although FIG. 1 shows two target devices 18a and 18b, the disclosure is not limited in this respect, and configuration 100 according to some embodiments may include any suitable number of target devices 18. According to some embodiments, configuration 100 includes target devices 18 that share the same I2C address and are assembled to a same extended I2C bus 15 with flipped I2C signals: (SCL, SDA), (SDA, SCL). For example, in some embodiments, target devices 18 may be or include multiple digital magnetometer sensors in an EM curve-tracked endoscope, as described, for example, in U.S. Pat. No. 11,712,309. In some embodiments, the multiple digital magnetometer sensors may share a same I2C address, for example, in cases where the number of sensors on the bus exceeds the number of different I2C addresses. In some embodiments, multiple sensors, for example six sensors, of a single I2C address may share a same I2C extended bus, which may consist of two or more I2C signals, multiplexed between the target devices.

For example, suppose two target devices 18a and 18b, for example, two digital magnetometer sensors, share the same I2C target address and are assembled to a same extended I2C bus 15 in a multiplexed configuration, as depicted in FIG. 1. In some embodiments, each target device 18 includes an SCL port and an SDA port. Target device 18a may be connected to I2C signals (SCL, SDA), i.e., in a “regular” configuration, such that the SCL signal wire is connected to the SCL port, and the SDA signal wire is connected to the SDA port. Target device 18b may be connected to I2C signals (SDA, SCL), i.e. in a “flipped” configuration, such that the SCL signal wire is connected to the SDA port, and the SDA signal wire is connected to the SCL port. Accordingly, the SCL signal serves as the I2C master clock for target device 18a, but serves as the I2C data signal for target device 18b, and the SDA signal serves as the I2C data signal for target device 18a, but serves as the I2C master clock for target device 18b.

Although target devices 18 share the same I2C address, the I2C controller device 10 may choose to address target device 18a and target device 18b separately, for example by internally switching the functionality of its SCL, SDA signals. Controller device 10 may then address target device 18a by treating SCL as I2C master clock and SDA as I2C data “regularly”, e.g. similarly to signal allocation of a standard I2C bus, and may address target device 18b in a “flipped” manner by switching the functionality of SCL, SDA, thus treating SDA as I2C master clock and SCL as I2C data.

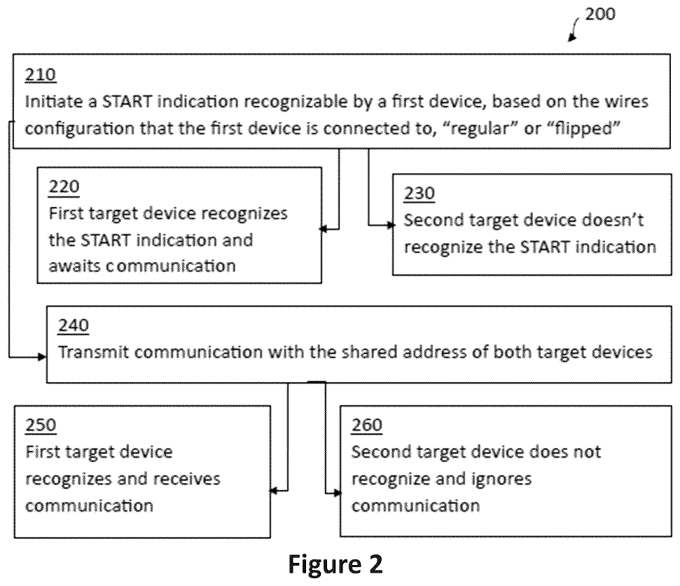

Reference is now made to FIG. 2, which is a schematic flowchart illustrating a method 200 for communication in an extended multiplexed inter-integrated circuit bus configuration 100 for digital endoscopy, according to some embodiments of the present disclosure. Target devices 18a and 18b may each be addressed individually by I2C controller 10, by switching the functionality of the SCL, SDA signals. For example, when controller device 10 addresses target device 18a, it treats SCL, SDA, “regularly”, e.g., similarly to a standard I2C bus, for example by treating SCL as I2C master clock and SDA as I2C data. When controller device 10 addresses target device 18b, it treats SCL, SDA, in a “flipped” manner, e.g., by treating SCL as I2C data and SDA as I2C master clock.

As indicated in block 210, controller device 10 may initiate a START indication recognizable by a first target device 18, based on the wires configuration that the first device is connected to, for example “regular” or “flipped”. For example, controller device 10 may write a START condition on the bus 15 by a falling edge of SDA while SCL is held HIGH. As indicated in block 220, the first target device recognizes the START indication and awaits communication and address. For example, target device 18a detects the START condition and expects the I2C controller 10 to communicate the READ/WRITE indication bit and the I2C target address. As indicated by block 230, the second target device doesn't recognize the START indication. For example, target device 18b sees the START condition as a falling edge of its clock “SCL” (which is actually the SDA signal of controller 10 while its data “SDA” (the SCL signal of controller 10) is HIGH, which is not detected as a START/STOP condition by target device 18b. Therefore, the START condition is detected by target device 18a as START, and is ignored by target device 18b. A STOP condition is indicated on the I2C bus, for example, by a rising edge of SDA while SCL is held HIGH. Accordingly, a STOP condition initiated by controller device 10 is detected by target device 18a as STOP, and is ignored by target device 18b.

As indicated in block 240, controller device 10 may transmit communication with the shared address of both target devices 18. As indicated in block 250, the first target device recognizes and receives communication. As indicated in block 260, the second target device does not recognize and ignores communication. For example, when data is written by controller device 10 on the I2C bus, target device 18a expects the data and receives it, and target device 18b ignores it. For example, when “0” is written (by holding SDA low and a rising edge of SCL) target device 18b ignores the event as its “SCL” (master's SDA) is held LOW. However, when “1” is written (by holding SDA high and a rising edge of SCL) target device 18b interprets that as a STOP condition on the bus, which may be followed by a START condition on the bus on the next falling edge of SCL. In any case, target device 18b sees a stream of START/STOP conditions without more than 2-3 READ/WRITE bits in between, such that target device 18b is not addressed on the flipped I2C bus 15. In this embodiment, target device 18b keeps silent (i.e., leaves its data line in HiZ state) while controller device 10 addresses target device 18a, and target device 18a keeps silent when controller device 10 addresses target device 18b, for example, by switching the functionality of its SCL, SDA lines.

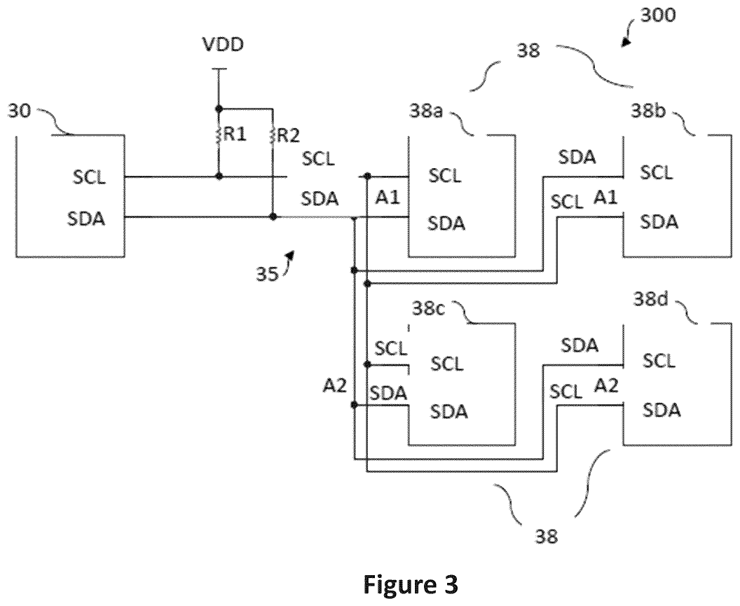

In other embodiments, multiple I2C target device addresses may be available. Reference is now made to FIG. 3, which is a schematic illustration of an extended multiplexed inter-integrated circuit bus configuration 300 for digital endoscopy, according to some embodiments of the present disclosure. For example, configuration 300 may include an extended bus 35, having, for example, two different 7-bit I2C addresses: A1 and A2. According to some embodiments of the present disclosure, extended multiplexed I2C bus 35 enables connection and separate communication of a controller device 30 with, for example, four target devices, for example four sensors. According to some embodiments, target devices 38a and 38b share the same I2C address A1, and target devices 38c and 38d share the same I2C address A2.

In such embodiments, target devices 38a and 38b may share the same I2C address A1 and may be connected to the extended I2C bus with a multiplexed configuration. For example, target device 38a is connected with a “regular” configuration (SCL, SDA), and target device 38b is connected with a “flipped” configuration (SDA, SCL). Target devices 38c and 38d may share the same I2C address A2 and may be similarly connected to the extended I2C bus with a multiplexed configuration. For example, target device 38c is connected with a “regular” configuration (SCL, SDA), and target device 38d is connected with a “flipped” configuration (SDA, SCL). When controller device 30 addresses target device 38a it may use bus 35 “regularly”, e.g., similarly to a standard I2C bus, for example by treating SCL as I2C master clock and SDA as I2C data, so that the “flipped” target devices 38b and 38d ignore the signals, as described herein, for example, with reference to FIG. 2. Then, controller device 30 may address target device 38a by specifying its I2C address A1 in the communication, such that target device 38c ignores the communication. Similarly, controller device 30 may address target device 38c by using bus 35 “regularly”, with I2C address A2. It can address target device 38b by using the “flipped” configuration, with I2C address A1, and finally, it can address target device 38d by using the “flipped” configuration, with I2C address A2. By choosing a SCL/SDA multiplexing configuration, and an I2C address (either A1 or A2), controller device 30 has a total of four unique choices for communication on the extended multiplexed I2C bus 35 to communicate with each of the four target devices 38 individually.

In some other embodiments, the I2C bus can be further extended by adding additional clock/data wires to the bus. For example, an additional “SDB” wire can be added to the bus for a total of three I2C wires: SCL, SDA, SDB, each of which is pulled-up by a pull-up resistor (for example, 10 kOhm, 4.7 kOhm, 1.5 kOhm, 1 kOhm, 700 Ohm, 500 Ohm or of any other suitable value). By selecting two of the three possible I2C wires, designating one wire as clock (SCL) and the second as data (SDA), the I2C controller is then able to communicate individually with each of a plurality of I2C target devices which are connected to any pair of those three possible I2C wires.

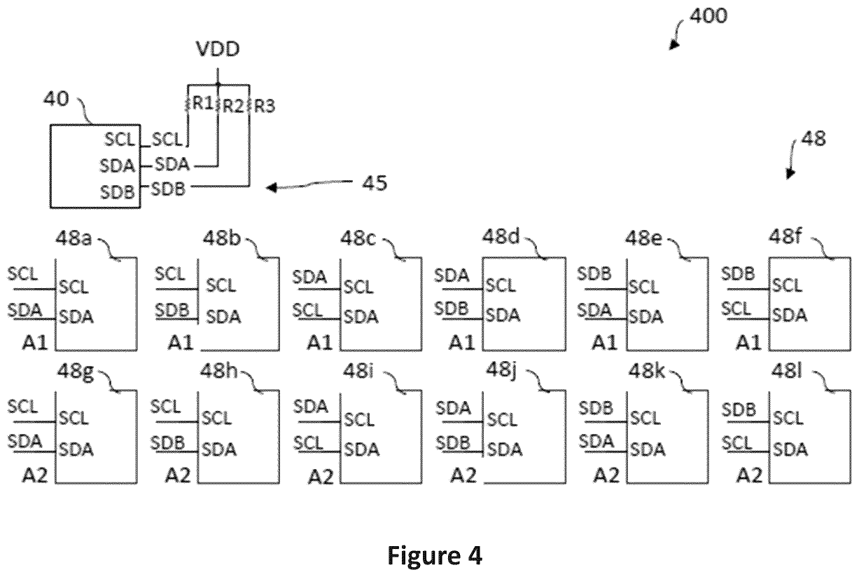

Reference is now made to FIG. 4, which is a schematic illustration of an extended multiplexed inter-integrated circuit bus configuration 400 for digital endoscopy, according to some embodiments of the present disclosure. In some embodiments, a plurality, for example twelve, of target devices 48 are connected to controller device 40 by the same I2C extended bus 45, which includes three wires: SCL, SDA, SDB, in a multiplexed configuration 400. Target devices 48 are provided in two different I2C addresses: A1, A2. These target devices can be or include, for example, digital magnetometer sensors, assembled on an FPC, according to some embodiments. Target devices 48a-48f may share the same I2C address A1, and Target devices 48g-481 may share the same I2C address A2. Each target device of a same I2C address is connected in a uniquely multiplexed configuration, as not to conflict with other target devices of the same address, as depicted, for example, in FIG. 4. For three extended I2C wires there may be six unique pair combinations which can be selected, these are: (SCL,SDA), (SDA,SCL), (SCL,SDB), (SDB,SCL), (SDB,SDA), (SDA,SDB). With two different I2C addresses A1, A2, twelve target devices 48 may be connected uniquely to controller device 40 by extended I2C bus 45, so as to enable each target devices 48 to be addressed individually by controller device 40.

When I2C control device 40 communicates with a certain multiplexed configuration “SCL”, “SDA”, according to some embodiments, it may place the third, unused, I2C extended wire at a HiZ (HIGH logic due to the pull-up resistor) state, or a LOW logic state. In any such case, any of the I2C target devices which are connected to the third (unused) wire will not respond to any communication, since none of the generated communication is meaningful enough for these target devices to initiate communication. For example, if the third wire is connected to the SCL port of a certain target device, then this certain target device will not see any communication, as the signal at its SCL port is not oscillating. On the other hand, if the third wire is connected to the SDA port of a certain target device, then the target device may only potentially see static HIGH/LOW bit READ/WRITE's (all zeros or all ones), which does not include any of the target addresses, and therefore the certain device ignores the communication.

In some other embodiments, an extended I2C bus may consist of W≥2 extended I2C wires (each of which is pulled up by a pull-up resistor). The I2C target devices may be available in M different I2C target addresses. The number of different multiplexed configurations is then W·(W−1) (different combinations to choose “SCL”, “SDA” out of the W possible wires). But each of these choices can be connected to a target device of M different addresses. This allows for a total of N(W, M)=M·W·(W−1) target devices to be connected on the extended I2C bus and addressed individually by the I2C controller device.

For example, for N(3,2)=12, N(3,4)=24, N(2,4)=8, N(4,2)=24, so for example, for an extended multiplexed 4-wire I2C bus (SCL, SDA, “SDB”, “SDC”), and with two different I2C target addresses, a total of 24 target devices can share the same bus and be addressed individually by the controller device. This enables, for example, placing 24 digital magnetometer sensors on an extended 4-wire I2C bus, with the only addition of two extra extended I2C wires, such that the extended I2C bus remains relatively small in footprint, which is potentially beneficial for endoscopy, according to some embodiments.

In most MCU's (Microcontroller Unit), there is only a limited amount of pin configurations for the hardware-supported I2C interface. Normally, it is not possible to configure an MCU to designate each pin to a different I2C signal. For example, in a standard MCU, pins 18, 19 may be designated for I2C SDA, SCL signals respectively. There may be a second set of supported I2C pin configuration, for example, pins 16, 17 may be designated for I2C SCL, SDA signals respectively. Choosing between the different pins configuration may be done in firmware/software. However, for the extended multiplexed I2C bus described herein, the I2C controller device needs to rapidly change pins functionality in order to address differently wired I2C target devices on the extended bus. For example, for a 3-wire extended I2C configuration (SCL, SDA, SDB), the I2C controller device needs to rapidly switch between pairs of SCL, SDA, SDB in I2C functionality, designating two of which as the I2C SCL, SDA signals. In order to achieve this, according to some embodiments, firmware/software I2C code can be used to communicate over the extended I2C bus in firmware/software, in an I2C “bit banging” technique. In another embodiment, an electrical mux component can be placed on the hardware I2C pins of the MCU, to select different pairs from the extended I2C signals. The mux can be configured in firmware/software by the MCU before initiating communication with one of the I2C slave devices on the extended bus. In another embodiment, an FPGA/ASIC chip can be used to perform the I2C communication on the extended bus. In this embodiment the FPGA/ASIC can perform the I2C multiplexing in its internal gates based on its configuration such that it designates each of its pins with a specific I2C functionality (SCL/SDA) based on the I2C target device that it needs to communicate with, according to some embodiments.

In some embodiments, the controller communicates in parallel with two or more target devices which are connected through different data signals but which may share a same clock signal. For example, two target devices may share the same SCL line, but may connect to a controller through separate I2C data lines: SDA, SDB respectively, in an extended I2C bus configuration as described herein. The controller may then initiate communication with both target devices simultaneously by generating a clock and a START condition on both SDA, SDB simultaneously. This can be achieved, for example, using an FPGA/ASIC which can generate synchronized communication on multiple pins. The two target devices then may respond to a READ request and output their data simultaneously, synchronized to the shared SCL clock, and the controller can read the output data on both its pins (SDA, SDB). Communicating with two or more devices simultaneously in an extended I2C bus configuration can potentially reduce the total communication time of the controller with all target devices, by making some of the communication parallel rather than sequential.

The extended multiplexed I2C bus described herein can support multiple I2C target devices on an extended I2C bus, with the optional addition of just a few extended I2C wires, to provide a communication bus with many I2C target devices (for example 24), some of which potentially share a same I2C address, with just a few I2C extended wires (for example, four wires), according to some embodiments. The extended I2C bus can be designed on an FPC and embedded in elongated devices such as an endoscope, for example, by wrapping the FPC inside the endoscope, for example, to provide communication with many digital magnetometers (for example, twelve), some of which potentially share a same I2C address, sharing a same extended I2C digital bus, and without significantly increasing the outer diameter of the endoscope, which is potentially beneficial for endoscopy, according to some embodiments.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for communication in an extended multiplexed inter-integrated circuit bus configuration, the system comprising:
a. an inter-integrated circuit bus including a plurality of wires;
b. a plurality of target devices, each includes at least two ports of respective two types; and
c. a controller device, communicatively connected to the plurality of target devices by an extended multiplexed configuration of the inter-integrated circuit bus,
wherein a first group of at least two of the target devices are allocated to a first same target address, wherein each of the target devices of the first group is connected to the bus by a different wire configuration of which wire is connected to which port type.

2. The system of claim 1, wherein the controller device is configured to:
a. initiate a start indication recognizable by a first target device of the first group, and unrecognizable by the other target devices of the first group, based on the wire configuration by which the first device is connected to the bus; and
b. transmit a communication along with an indication of the first same target address of the first group.

3. The system of claim 1, wherein a second group of at least two of the target devices are allocated to a second same target address, wherein each of the target devices of the second group is connected to the bus by a different wire configuration of which wire is connected to which port type, wherein the controller device is configured to:
a. initiate a start indication recognizable by a first target device of the first group and a first target device of the second group, and unrecognizable by the other target devices of the first and second groups, based on the wire configuration by which the first devices is connected to the bus; and
b. transmit a communication along with an indication of the same target address of the first group.

4. The system of claim 1, wherein the target devices of the same group share the same target address and are assembled to the same extended bus, with respective different wire configurations, in which a clock signal wire and a data signal wire are flipped in a first configuration with respect to a second configuration.

5. The system of claim 1, wherein the target devices comprise digital magnetometer sensors in an electromagnetic curve-tracked endoscope.

6. The system of claim 1, wherein the extended bus has at least three wires, wherein the controller selects two of the three possible wires, designates one wire as clock signal and the second as data signal, and communicates individually with each of a plurality of the target devices which are connected to the selected two wires.

7. The system of claim 1, wherein the extended bus wires are each pulled-up by a pull-up resistor.

8. A method for communication in an extended multiplexed inter-integrated circuit bus configuration, the method comprising:
a. initiating by a controller device a start indication recognizable by a first target device of a first group of devices, the start indication is unrecognizable by other target devices of the first group, based on a wire configuration by which the first device is connected to the bus,
wherein the first group includes at least two target devices that are allocated to a first same target address, wherein each of the target devices of the first group is connected to the bus by a different wire configuration of which wire is connected to which port type, wherein each target device includes at least two ports of respective two types; and
b. transmitting a communication along with an indication of the first same target address of the first group.

9. The method of claim 8, wherein a second group of at least two of the target devices are allocated to a second same target address, wherein each of the target devices of the second group is connected to the bus by a different wire configuration of which wire is connected to which port type, the method comprising:
a. initiating a start indication recognizable by a first target device of the first group and a first target device of the second group, and unrecognizable by the other target devices of the first and second groups, based on the wire configuration by which the first devices is connected to the bus; and
b. transmitting a communication along with an indication of the same target address of the first group.

10. The method of claim 8, wherein the target devices of the same group share the same target address and are assembled to the same extended bus, with respective different wire configurations, in which a clock signal wire and a data signal wire are flipped in a first configuration with respect to a second configuration.

11. The method of claim 8, wherein the target devices comprise digital magnetometer sensors in an electromagnetic curve-tracked endoscope.

12. The method of claim 8, wherein the extended bus has at least three wires, the method comprising selecting two of the three possible wires, designating one wire as clock signal and the second as data signal, and communicating individually with each of a plurality of the target devices which are connected to the selected two wires.

13. The method of claim 8, wherein the extended bus wires are each pulled-up by a pull-up resistor.
